(19) United States
(12) Patent Application Publication
(10) Pub. No.: US 2013/0274829 A1
(43) Pub. Date: Oct. 17, 2013

(54) NEUROSTIMULATION DEVICE HAVING FREQUENCY SELECTIVE SURFACE TO PREVENT ELECTROMAGNETIC INTERFERENCE DURING MRI

(71) Applicant: BOSTON SCIENTIFIC NEUROMODULATION CORPORATION, (US)

(72) Inventors: Gaurav Gupta, Valencia, CA (US); Kiran Gururaj, Valencia, CA (US)

(73) Assignee: BOSTON SCIENTIFIC NEUROMODULATION CORPORATION, Valencia, CA (US)

(21) Appl. No.: 13/864,071

(22) Filed: Apr. 16, 2013

Related U.S. Application Data
(60) Provisional application No. 61/625,208, filed on Apr. 17, 2012.

(51) Int. Cl. A61N 1/372

(52) U.S. Cl. CPC A61N 1/37217
USPC 607/61

(57) ABSTRACT

An implantable medical device comprises an antenna configured for wirelessly receiving energy of a first frequency from an external device, electronic circuitry configured for performing a function in response to the receipt of the received energy, and a biocompatible housing containing the electronic circuitry and antenna. The housing includes a substrate structure and a two-dimensional array of elements disposed on the substrate structure. The array of elements and substrate structure are arranged in a manner that creates a frequency selective surface capable of reflecting at least a portion of energy of a second frequency incident on the housing, while passing at least a portion of energy of the first frequency incident on the housing to the antenna.

### RELATED APPLICATION DATA

The present application claims the benefit under 35 U.S.C. §119 to U.S. provisional patent application Ser. No. 61/625,208, filed Apr. 17, 2012. The foregoing application is hereby incorporated by reference into the present application in its entirety.

### FIELD OF THE INVENTION

The present invention relates to tissue stimulation systems, and in particular, MRI-compatible neurostimulators.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., Arrhythmias). Spinal Cord Stimulation (SCS) systems have long been accepted as a therapeutic modality for the treatment of chronic pain syndromes, and the application of tissue stimulation has begun to expand to additional applications such as Angina Pectoralis and Incontinence. Deep Brain Stimulation (DBS) has also been applied therapeutically for well over a decade for the treatment of refractory chronic pain syndromes, and DBS has also recently been applied in additional areas such as movement disorders and Epilepsy. Further, in recent investigations Peripheral Nerve Stimulation (PNS) systems have demonstrated efficacy in the treatment of chronic pain syndromes and incontinence, and a number of additional applications are currently under investigation. Furthermore, Functional Electrical Stimulation (FES) systems such as the Freehand system by NeuroControl (Cleveland, Ohio) have been applied to restore some functionality to paralyzed extremities in spinal cord injury patients.

Each of these implantable neurostimulation systems typically includes at least one stimulation lead implanted at the desired stimulation site and an Implantable Pulse Generator (IPG) implanted remotely from the stimulation site, but coupled either directly to the stimulation lead(s) or indirectly to the stimulation lead(s) via one or more lead extensions. Thus, electrical pulses can be delivered from the neurostimulator to the electrodes carried by the stimulation lead(s) to stimulate or activate a volume of tissue in accordance with a set of stimulation parameters and provide the desired efficacious therapy to the patient.

The neurostimulation system may further comprise a handheld Remote Control (RC) to remotely instruct the neurostimulator to generate electrical stimulation pulses in accordance with selected stimulation parameters. The RC may, itself, be programmed by a technician attending the patient, for example, by using a Clinician's Programmer (CP), which typically includes a general purpose computer, such as a laptop, with a programming software package installed thereon. The RC and CP wirelessly communicate with the IPG using an RF signal of a specific frequency or range of frequencies (e.g., at a center frequency of 125 KHz) that is received by one or more telemetry coils in the IPG.

The neurostimulation system may also include an external charger capable of wirelessly conveying energy at a specific frequency or range of frequencies (e.g., at a center frequency of 84 KHz) from an alternating current (AC) charging coil in the external charger to a reciprocal AC coil located in the IPG. The energy received by the charging coil located on the IPG can then be used to directly power the electronic circuitry contained within the IPG, or can be stored in a rechargeable battery within the IPG, which can then be used to power the electronic circuitry on-demand.

IPGs are routinely implanted in patients who are in need of Magnetic Resonance Imaging (MRI). Thus, when designing implantable neurostimulation systems, consideration must be given to the possibility that the patient in which neurostimulator is implanted may be subjected to electro-magnetic forces generated by MRI scanners, which may potentially cause damage to the neurostimulator as well as discomfort to the patient.

In particular, in MRI, spatial encoding relies on successively applying magnetic field gradients. The magnetic field strength is a function of position and time with the application of gradient fields throughout the imaging process. Gradient fields typically switch gradient coils (or magnets) ON and OFF thousands of times in the acquisition of a single image in the presence of a large static magnetic field. Present-day MRI scanners can have maximum gradient strengths of 100 mT/m and much faster switching times (slew rates) of 150 mT/m/ms, which is comparable to stimulation therapy frequencies. Typical MRI scanners create gradient fields in the range of 100 Hz to 30 KHz, and Radio Frequency (RF) fields of 64 MHz for a 1.5 Tesla scanner and 128 MHz for a 3 Tesla scanner.

In an MRI environment, the radiated RF fields may impinge on an IPG and cause different types of problems, including damage to the electronic circuitry in the IPG and patient discomfort due to heating of the IPG. For example, the RF fields may create eddy currents on the larger conductive surfaces of the IPG, such as the surface of the housing and the battery. The eddy currents, in turn, create thermal energy that may damage the battery as well cause discomfort to the patient or even damage to the tissue surrounding the IPG. The radiated RF field may also be picked up by charging or telemetry coils within the IPG, which my result in damage to the electronics coupled to these coils. Of course, not all radiated energy is harmful to the IPG; for example, the energy transmitted by the RC, CP and/or external charger to convey programming information or charge the IPG.

There, thus, remains a need to prevent heating of the IPG during an MRI, while allowing energy used to communicate and/or charge an IPG.

### SUMMARY OF THE INVENTION

In accordance with the present inventions, an implantable medical device is provided. The medical device comprises an antenna configured for wirelessly receiving energy of a first frequency from an external device, electronic circuitry configured for performing a function (e.g., programming and/or charging the medical device) in response to the receipt of the received energy, and a biocompatible housing containing the electronic circuitry and antenna.

The housing includes a substrate structure and a two-dimensional array of elements disposed on the substrate structure. The array of elements may be periodic, and the elements may be identical in shape. Each of the elements may be, e.g., one of linear dipole, crossed dipole, loop, and a bow-tie. Each of the elements may have an impedance load. The impedance load may be adjustable, in which case, the implantable medical device may further comprise an electronic controller coupled to the impedance load. The electronic controller may be configured for generating a signal that dynamically adjusts the impedance load. In one embodiment, one of the substrate structure and the array of elements is composed of a dielectric material (e.g., ceramic or plastic), and the other of the substrate structure and the array of elements is composed of an electrically conductive material (e.g., metal). The array of elements and substrate structure are arranged in a manner that creates a Frequency Selective Surface (FSS) capable of reflecting at least a portion of energy of a second frequency (e.g., greater than 10 MHz) incident on the housing, while passing at least a portion of energy of the first frequency (e.g., less than 200 KHz) incident on the housing to the antenna.

In one embodiment, the transmission coefficient for the energy of the first frequency incident on the housing is greater than 0.5, and the reflection coefficient for the energy of the second frequency incident on the housing is greater than 0.5. In another embodiment, the transmission coefficient for the energy of the first frequency incident on the housing is greater than 0.75, and the reflection coefficient for the energy of the second frequency incident on the housing is greater than 0.75.

In another embodiment, the medical device further comprises a battery contained within the housing. The battery may include another substrate structure and another two-dimensional array of elements disposed on the other substrate structure, in which case, the other array of elements and other substrate structure may be arranged in a manner that creates a frequency selective surface capable of reflecting at least a portion of energy of a third frequency (which may be the same as the second frequency) incident on the battery, while passing at least a portion of the energy of the second frequency incident on the battery to the antenna.

In still another embodiment, the medical device further comprises a lead coupled to the electronic circuitry. The lead includes a tubular substrate structure and another two-dimensional array of elements disposed on the tubular substrate structure, in which case, the other array of elements and other substrate structure may be arranged in a manner that creates a frequency selective surface capable of reflecting at least a portion of energy of a third frequency (which may be the same as second frequency) incident on the lead.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of a Spinal Cord Stimulation (SCS) system constructed in accordance with one embodiment of the present inventions;

FIG. 2 is a plan view of the SCS system of FIG. 1 in use within a patient;

FIG. 3 is a plan view of an implantable pulse generator (IPG) and three percutaneous stimulation leads used in the SCS system of FIG. 1;

FIG. 4 is a plan view of an implantable pulse generator (IPG) and a surgical paddle lead used in the SCS system of FIG. 2;

FIGS. 5a and 5b are plan views of different types of frequency selective surfaces that can be incorporated into the housing of the IPG of FIGS. 3 and 4;

FIG. 6a-6d are cross-sectional views of different housings that can be used for the IPG of FIGS. 3 and 4;

FIGS. 7a-7d are plan views of different elements that can be used to create a frequency selective surface for the housing of the IPG of FIGS. 3 and 4;

FIG. 8 is a circuit diagram of an impedance load adjustment circuit that can be used to adjust the frequency selective surface for the housing of the IPG of FIGS. 3 and 4;

FIG. 9 is a perspective view of one embodiment of a battery contained within the IPG of FIGS. 3 and 4; and

FIG. 10 is a perspective view of one embodiment of a stimulation lead of FIG. 3.

### DETAILED DESCRIPTION OF THE EMBODIMENTS

The description that follows relates to a Spinal Cord Stimulation (SCS) system. However, it is to be understood that the while the invention lends itself well to applications in SCS, the invention, in its broadest aspects, may not be so limited. Rather, the invention may be used with any type of implantable electrical circuitry used to stimulate tissue. For example, the present invention may be used as part of a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neural stimulator configured to treat urinary incontinence, sleep apnea, shoulder sublaxation, headache, etc.

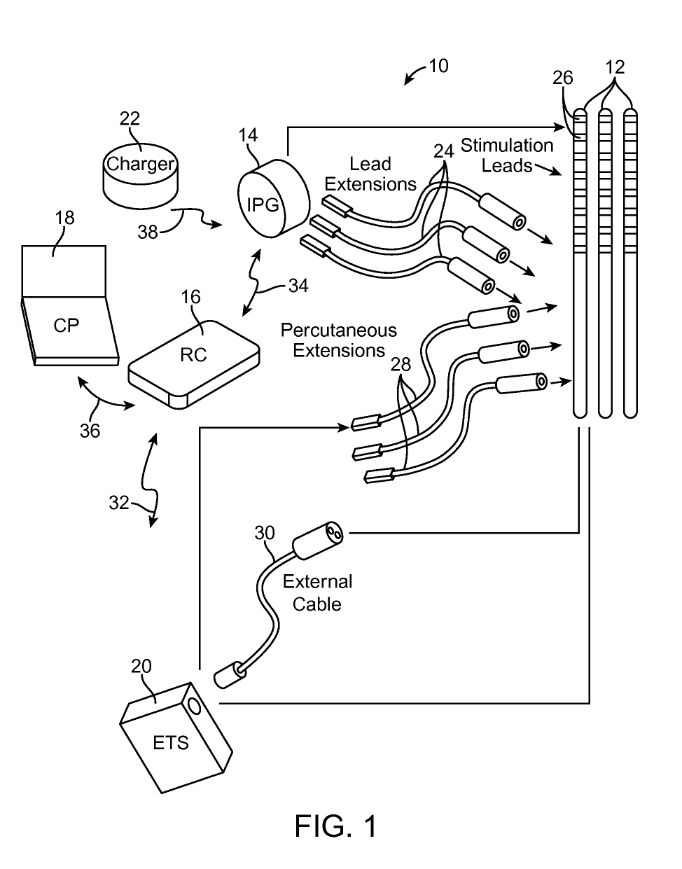

Turning first to FIG. 1, an exemplary spinal cord stimulation (SCS) system 10 generally includes one or more (in this case, three) implantable stimulation leads 12, a pulse generating device in the form of an implantable pulse generator (IPG) 14, an external control device in the form of a remote controller RC 16, a clinician's programmer (CP) 18, an external trial stimulator (ETS) 20, and an external charger 22.

The IPG 14 is physically connected via one or more lead extensions 24 to the stimulation leads 12, which carry a plurality of electrodes 26 arranged in an array. The stimulation leads 12 are illustrated as percutaneous leads in FIG. 1, although as will be described in further detail below, a surgical paddle lead can be used in place of the percutaneous leads. As will also be described in further detail below, the IPG 14 includes pulse generation circuitry that delivers electrical stimulation energy in the form of a pulsed electrical waveform (i.e., a temporal series of electrical pulses) to the electrode array 26 in accordance with a set of stimulation parameters.

The ETS 20 may also be physically connected via the percutaneous lead extensions 28 and external cable 30 to the stimulation leads 12. The ETS 20, which has similar pulse generation circuitry as the IPG 14, also delivers electrical stimulation energy in the form of a pulse electrical waveform to the electrode array 26 accordance with a set of stimulation parameters. The major difference between the ETS 20 and the IPG 14 is that the ETS 20 is a non-implantable device that is used on a trial basis after the stimulation leads 12 have been implanted and prior to implantation of the IPG 14, to test the responsiveness of the stimulation that is to be provided. Thus, any functions described herein with respect to the IPG 14 can likewise be performed with respect to the ETS 20.

The RC 16 may be used to telemetrically control the ETS 20 via a bi-directional RF communications link 32. Once the IPG 14 and stimulation leads 12 are implanted, the RC 16 may be used to telemetrically control the IPG 14 via a bi-directional RF communications link 34. Such control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. As will be described in further detail below, the CP 18 provides clinician detailed stimulation parameters for programming the IPG 14 and ETS 20 in the operating room and in follow-up sessions.

The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown). The clinician detailed stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be described herein. Details of exemplary embodiments of these devices are disclosed in U.S. Pat. No. 6,895,280, which is expressly incorporated herein by reference.

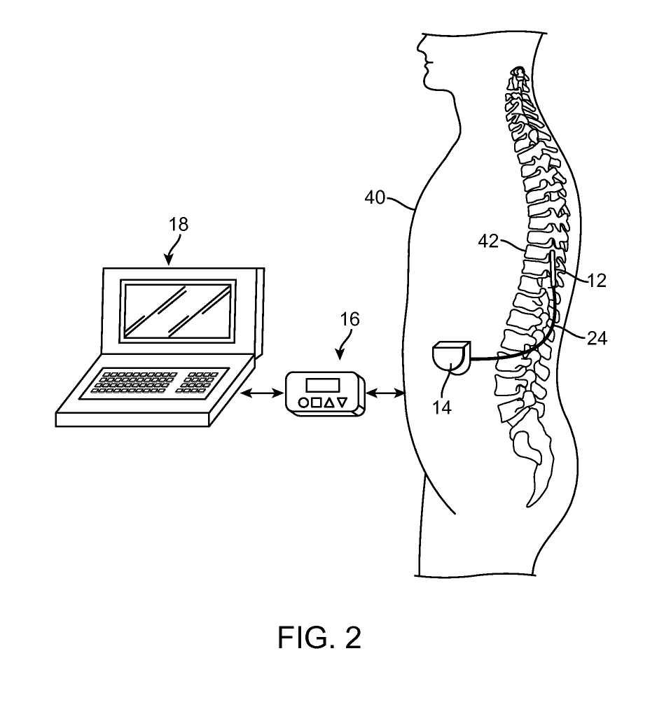

As shown in FIG. 2, the stimulation leads 12 are implanted within the spinal column 42 of a patient 40. The preferred placement of the electrode leads 12 is adjacent, i.e., resting near, the spinal cord area to be stimulated. Due to the lack of space near the location where the electrode leads 12 exit the spinal column 42, the IPG 14 is generally implanted in a surgically-made pocket either in the abdomen or above the buttocks. The IPG 14 may, of course, also be implanted in other locations of the patient's body. The lead extensions 24 facilitate locating the IPG 14 away from the exit point of the electrode leads 12. As there shown, the CP 18 communicates with the IPG 14 via the RC 16.

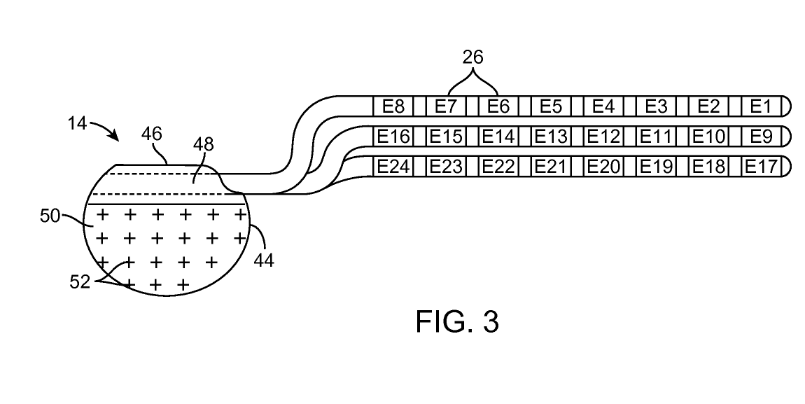

Referring now to FIG. 3, the external features of the stimulation leads 12 and the IPG 14 will be briefly described. Each of the stimulation leads 12 has eight electrodes 26 (respectively labeled E1-E8, E9-E16, and E17-E24). The actual number and shape of leads and electrodes will, of course, vary according to the intended application. Further details describing the construction and method of manufacturing percutaneous stimulation leads are disclosed in U.S. patent application Ser. No. 11/689,918, entitled “Lead Assembly and Method of Making Same,” and U.S. patent application Ser. No. 11/565,547, entitled “Cylindrical Multi-Contact Electrode Lead for Neural Stimulation and Method of Making Same,” the disclosures of which are expressly incorporated herein by reference.

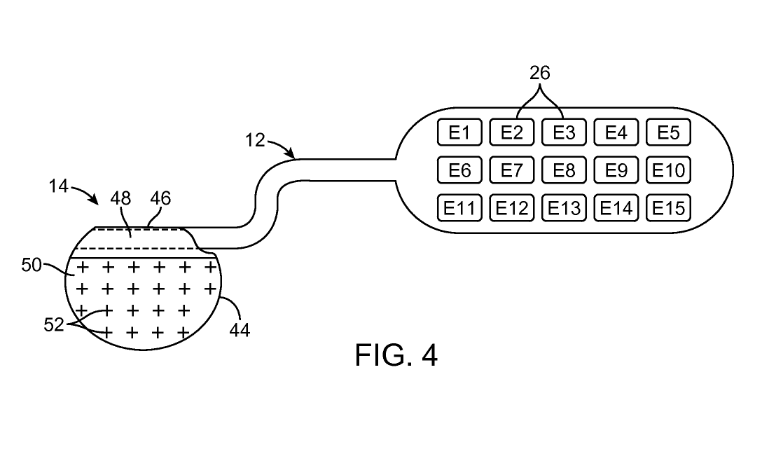

Alternatively, as illustrated in FIG. 4, the stimulation lead 12 takes the form of a surgical paddle lead on which electrodes 26 are arranged in a two-dimensional array in three columns (respectively labeled E1-E5, E6-E10, and E11-E15) along the axis of the stimulation lead 12. In the illustrated embodiment, five rows of electrodes 26 are provided, although any number of rows of electrodes can be used. Each row of the electrodes 26 is arranged in a line transversely to the axis of the lead 12. The actual number of leads and electrodes will, of course, vary according to the intended application. Further details regarding the construction and method of manufacture of surgical paddle leads are disclosed in U.S. patent application Ser. No. 11/319,291, entitled “Stimulator Leads and Methods for Lead Fabrication,” the disclosure of which is expressly incorporated herein by reference.

In each of the embodiments illustrated in FIGS. 3 and 4, the IPG 14 comprises an outer case (or housing) 44 for housing the electronics and other components (described in further detail below). The outer case 44 forms a hermetically sealed compartment that protects the internal electronics from the body tissue and fluids, while permitting passage of electromagnetic fields used to transmit data and/or power. In some cases, the outer case 44 may serve as an electrode. The IPG 14 further comprises a connector 46 to which the proximal ends of the stimulation leads 12 mate in a manner that electrically couples the electrodes 26 to the internal electronics (described in further detail below) within the outer case 44. To this end, the connector 46 includes one or more ports (three ports 48 or three percutaneous leads or one port for the surgical paddle lead) for receiving the proximal end(s) of the stimulation lead(s) 12. In the case where the lead extensions 24 are used, the port(s) 48 may instead receive the proximal ends of such lead extensions 24.

The IPG 14 includes pulse generation circuitry that provides electrical conditioning and stimulation energy in the form of a pulsed electrical waveform to the electrode array 26 in accordance with a set of stimulation parameters programmed into the IPG 14. Such stimulation parameters may comprise electrode combinations, which define the electrodes that are activated as anodes (positive), cathodes (negative), and turned off (zero), percentage of stimulation energy assigned to each electrode (fractionalized electrode configurations), and electrical pulse parameters, which define the pulse amplitude (measured in milliamps or volts depending on whether the IPG 14 supplies constant current or constant voltage to the electrode array 26), pulse width (measured in microseconds), pulse rate (measured in pulses per second), and burst rate (measured as the stimulation on duration X and stimulation off duration Y).

Additional details concerning the above-described and other IPGs may be found in U.S. Pat. No. 6,516,227, U.S. Patent Publication No. 2003/0139781, and U.S. patent application Ser. No. 11/138,632, entitled “Low Power Loss Current Digital-to-Analog Converter Used in an Implantable Pulse Generator,” which are expressly incorporated herein by reference. It should be noted that rather than an IPG, the system 10 may alternatively utilize an implantable receiver-stimulator (not shown) connected to leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

Significantly, the outer case 44 is constructed in a manner that creates a Frequency Selective Surface (FSS) that, when exposed to electromagnetic radiation, generates a scattered wave with a prescribed frequency response. Thus, the FSS serves as a filter for electromagnetic energy, and in particular, is capable of reflecting at least a portion of energy at a first frequency (e.g., electromagnetic fields emitted during an MRI) that are incident on the case 44, while passing at least a portion of energy of a second frequency incident on the case 44 (e.g., programming signals or charging energy) to the necessary componentry contained in the case 44, e.g., an antenna, such as a coil for receiving programming signals and/or charging energy).

Preferably, the energy that is reflected is greater than 10 MHz, which will typically encompass typical RF frequencies used in MRI scanners (e.g., 64 MHz and 128 MHz), while the energy that is passed is less than 200 KHz, which will typically encompass RF frequencies used in programming signals and charging energy (e.g., 84 KHz and 125 KHz, respectively). It is preferable that a substantial amount of the energy at the first frequency be reflected, and that a substantial amount of the energy at the second frequency be passed. In an optional embodiment, the energy that is reflected is also less than 40 KHz, which will typically encompass typical gradient fields used in MRI scanners (e.g., 100 Hz to 30 KHz). For the reflection coefficient (i.e., the percentage of reflected energy divided by incident energy) is preferably greater than 0.5, and more preferably greater than 0.75, whereas the transmission coefficient (i.e., the percentage of transmitted energy divided by incident energy) is preferably greater than 0.5, and more preferably greater than 0.75.

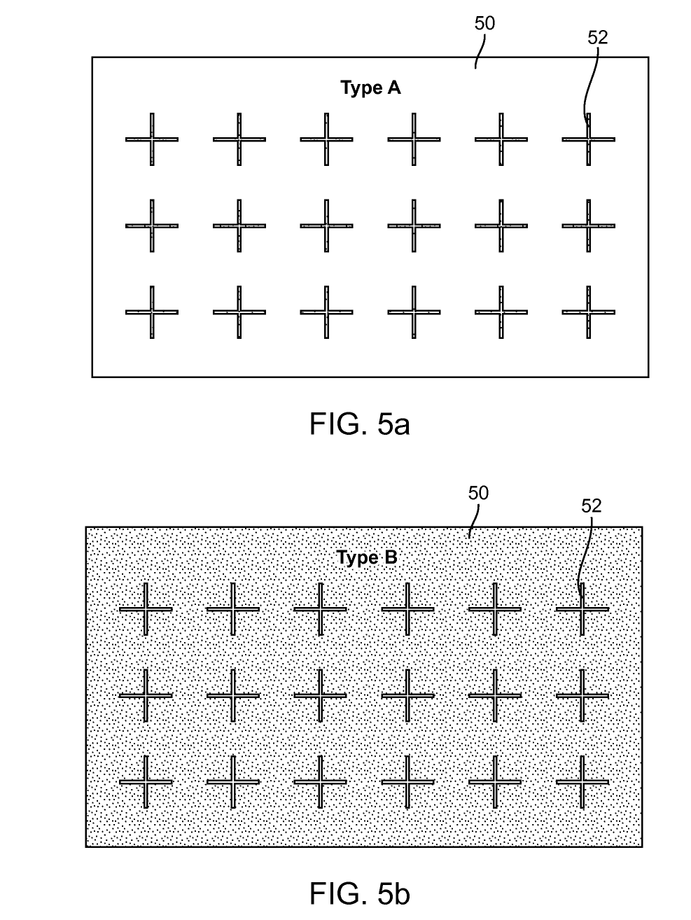

The case 44 includes a substrate structure 50 and a two-dimensional array of elements 52 disposed on the substrate structure 50, thereby creating the FSS, which can be generally of two types. In particular, a “Type A” FSS is shown in FIG. 5a, in which the substrate structure 50 is composed of a dielectric material, while the elements 52 are composed of an electrically conductive material. In FIG. 5b, a “Type B” FSS is shown, in which the substrate structure 50 is composed of an electrically conductive material, while the elements 52 are composed of a dielectric material. The dielectric material may be, e.g., ceramic or plastic, whereas the electrically conductive material, may be, e.g., metal, such as titanium.

The Type A surface has a complimentary response compared to Type B surface.

For example, if the element is a patch, the Type A FSS has a capacitive surface, and thus, exhibits a low-pass characteristic, such that the FSS passes energy at lower frequencies, while reflecting energy at high frequencies. The Type B FSS has an inductive surface, and thus, exhibits a low-pass characteristic, such that the FSS passes energy at lower frequencies, while reflecting energy at high frequencies. Thus, the Type A FSS is particularly useful to reflect the higher frequency MRI electromagnetic fields, while passing the lower frequency programming signals and/or charging energy, whereas the Type B FSS is particularly useful to reflect the undesirable energy associated with lower frequencies, while passing the higher frequency programming signals and/or charging energy.

In another example, if the element is a cross-dipole, it can be modeled as a shunt element, comprising of series inductor and capacitor between the input and output. At resonance, this will lead to a complete reflection, thereby giving the surface a band-stop response. Thus, the Type A FSS surface with cross dipoles will be particularly useful in reflecting the higher frequency MRI electromagnetic fields, while passing the lower frequency energy. On the other hand, the Type B FSS surface will have a band-pass response, and thus will be particularly useful to reflect the undesirable energy associated with lower frequencies, while passing the higher frequency programming signals and/or charging energy.

The reflection/transmission coefficient and frequencies of the energy that is reflected/transmitted depend upon the type of element 52 (e.g., size, shape, loading, and orientation), distance between the elements 52 in both directions (x- and y-directions), conductivity of the elements 52 (which increases the reflectivity), and whether which of the substrate structure 50 and elements 52 is composed of a dielectric material, and which one is composed of an electrically conductive material.

The effective length of the elements 52 is preferably a half-wavelength at the frequency of the energy intended to be reflected in the case of a Type A FSS, and a half-wavelength at the frequency of the energy intended to be passed in the case of a Type B FSS. In this case, the coupling between elements 52 and the incident electromagnetic energy nominally reaches its highest level at the fundamental frequency where the effective length of the elements 52 is a half wavelength. In order to decrease the size of the elements 52, metamaterial based FSS techniques described in Metamaterial-Inspired Frequency-Selective Surfaces, Farhad Bayatpur, University of Michigan (2009), which is expressly incorporated herein by reference, can be used. As a general rule, the greater the spacing between the elements 52 is, the narrower the bandwidth of the energy that is reflected or passed, and the less the spacing between the elements 52 is, the wider the bandwidth of the energy that is reflected or passed.

The substrate structure 50 and array of elements 52 may be arranged in any one or more of a variety of ways to create the FSS. In the preferred embodiment, the array of elements 52 repeat in a periodic fashion, and the elements 52 are identical in geometry and have a uniform distance between each other. The elements 52 may be disposed on the substrate structure 50 in any one of a variety of manners, depending on whether FSS is a Type A FSS or a Type B FSS.

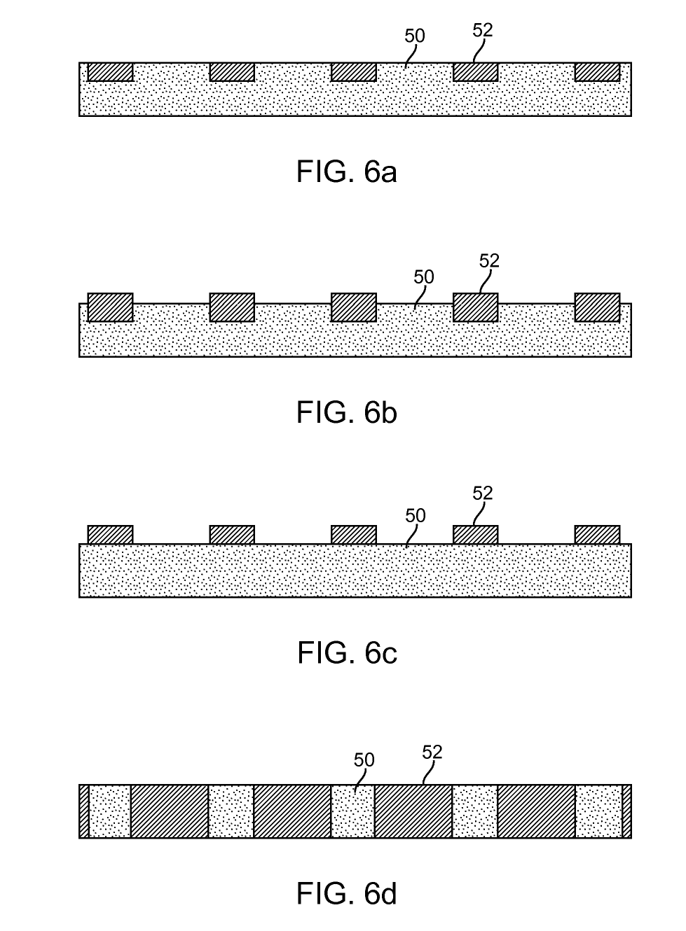

As one example shown in FIG. 6a, in the case of a Type A FSS, openings in the shape of the elements 52 can be partially formed in the dielectric substrate structure 50 in accordance with the desired pattern using a conventional technique, such as molding, and then the electrically conductive elements 52 can be disposed in the openings using a conventional technique, such as ion beam deposition. As shown in FIG. 6a, the electrically conductive elements 52 are flush with the surface of the dielectric substrate structure 50. Alternatively, as shown in FIG. 6b, the electrically conductive elements 52 may be raised above the surface of the dielectric substrate structure 50, thereby creating a relief pattern on the case 44. As another example shown in FIG. 6c, in the case of a Type A FSS, the electrically conductive elements 52 can be formed on the surface of the dielectric substrate structure 50 in the desired pattern, using a conventional technique, such as photochemical etching. As still another example shown in FIG. 6d, in the case of a Type B FSS, openings in the shape of the elements 52 can be completely formed through the dielectric substrate structure 50 in accordance with the desired pattern using a conventional technique, such as punching, and then the electrically conductive elements 52 can be disposed in the openings using a conventional technique, such as injection molding.

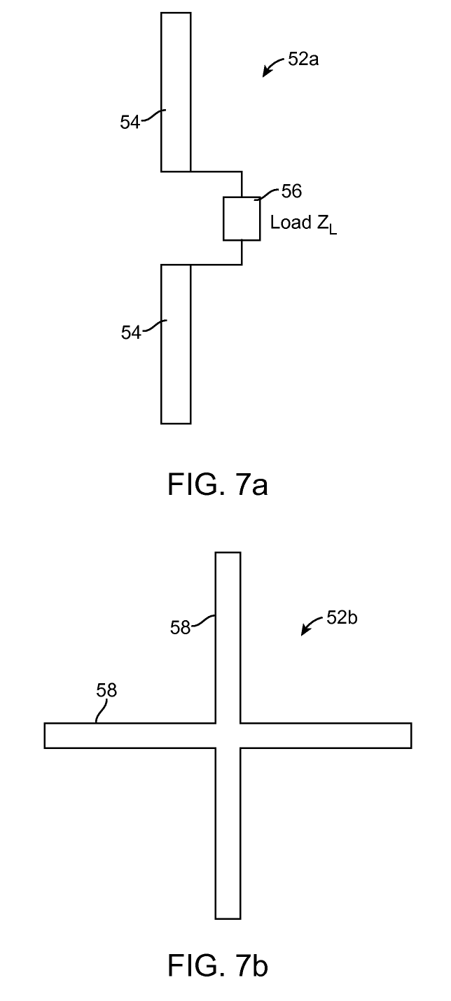

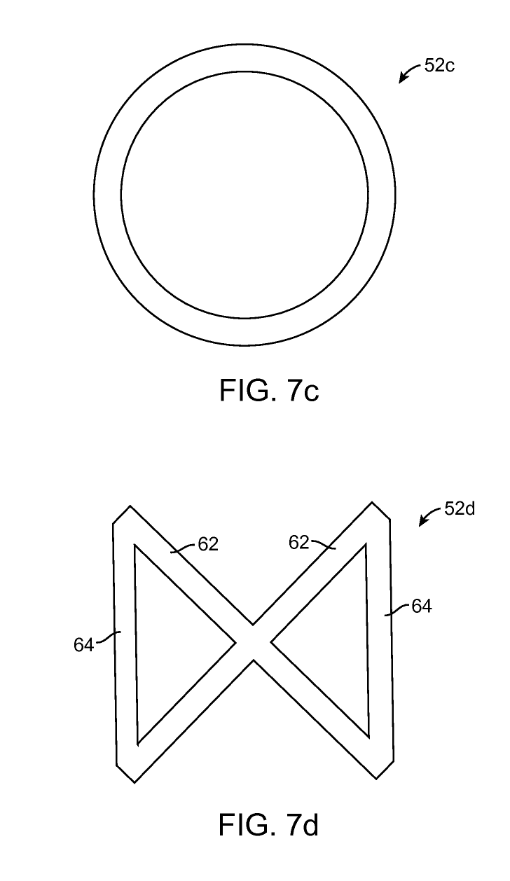

Referring to FIGS. 7a-7d, four different types of exemplary elements 52 will now be described. Notably, the types of elements that can be used in the present invention should not be limited to those illustrated in FIGS. 7a-7d. For example, the elements may take the form of rectangles (either solid or loops), Jerusalem crosses, three- or four-legged dipoles, meandering lines, zig-zags, etc.

In FIG. 7a, the element 52a takes the form of a loaded linear dipole. In this example, the element 52a includes two co-linear sub-elements 54 that are coupled to each other through an impedance load 56. Notably, in order to maximum the reflection coefficient of the FSS illustrated in FIG. 7a, it is preferable that the orientation of the electromagnetic waves in the energy designed to be reflected be oriented parallel with the orientation of the dipole element 52a.

Modification of the impedance load 56 will allow tuning of the FSS. For example, the inductance or capacitance of the impedance load 56 may be modified to change the frequency of the energy that is reflected/transmitted, while the resistance of the impedance lead 106 may be modified to change the bandwidth of the frequency range of the energy that is reflected/transmitted.

In FIG. 7b, the element 52b takes the form of a crossed-dipole. In this example, the element 52b includes two orthogonal sub-elements 58, which maximizes the reflection coefficient of the FSS for any orientation of the electromagnetic waves in the energy incident on the FSS. That is, any electromagnetic wave in the energy designed to be reflected will be broken into orthogonal components by the sub-elements 58.

In FIG. 7c, the element 52c takes the form of a loop. In this example, the circular element 52c interacts with the magnetic component of the electromagnetic wave in any orientation.

In FIG. 7d, the element 52d takes the form of a bow-tie. In this example, the element 52d includes two orthogonal sub-elements 60 and two parallel sub-elements 62 that couple the ends of the sub-elements 60 together. Due to the multiple sub-elements, the element 52d reflects energy over a broader frequency range.

Any of the elements 52 described above may be loaded by different lumped combination of components to create an impedance load, such as the impedance load 56 illustrated in FIG. 7a. Any of these impedance loads may advantageously be dynamically adjustable via signaling by an electronic controller, thereby providing a means to selectively reflect energy of different frequencies. For example, if a 1.5 Tesla MRI scanner is used, the impedance load can be modified, such that energy at a frequency of 64 MHz is reflected, whereas if a 3 Tesla MRI scanner is used, the impedance load can be modified, such that energy at a frequency of 128 MHz is reflected. A signal transmitted from the RC 16 or the CP 18 can prompt an electronic controller contained within the IPG 14 to adjust the impedance load.

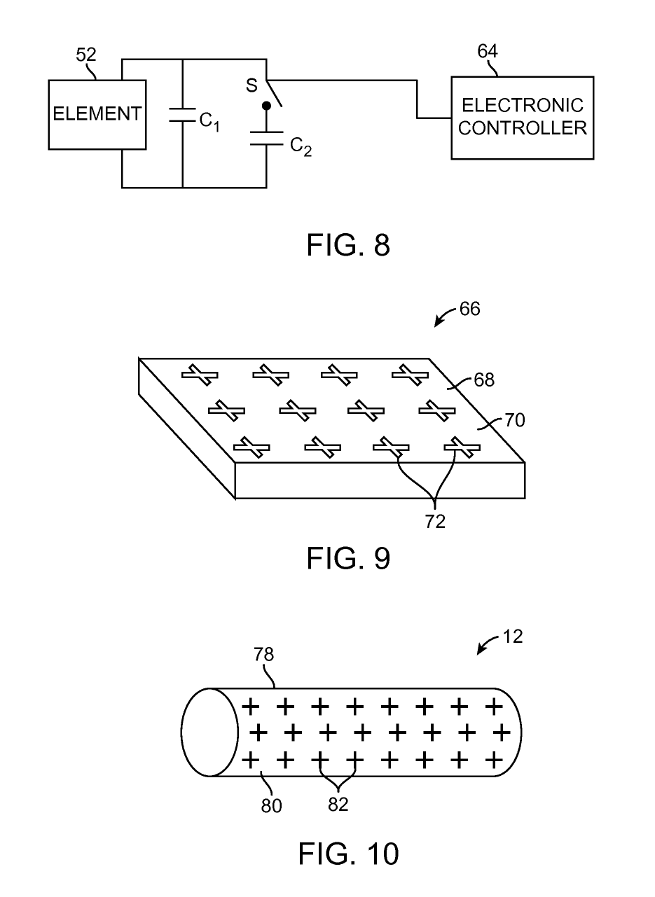

In one example illustrated in FIG. 8, an adjustable impedance load 62 comprises a pair of capacitors C1, C2 coupled in parallel to each other between terminals (not shown) of the respective element 52, with a switch S in series with the capacitor C2. The switch S may be selectively opened and closed in response to a signal generated by an electronic controller 64 contained within the IPG 14. When the switch S is open, only the capacitor C1 is coupled to the respective element 52, thereby reflecting energy at a higher frequency (e.g., 128 MHz). In contrast, when the switch S is closed, both capacitors C1 and C2 are coupled to the respective element 52, thereby reflecting energy at a lower frequency (e.g., 64 MHz).

Although the FSS has been described as being associated with the case 44 of the IPG 14, it should be appreciated that an FSS can be associated with other components of the IPG 14 or even other components of the SCS system 10.

For example, if the antenna is behind the battery, it may be useful to use an FSS for the battery in order to reflect MRI electromagnetic energy while passing programming signals and/or charging energy to the antenna. For example, referring to FIG. 9, a battery 66 may comprise a case 68 (or housing), which includes a substrate structure 70 and a two-dimensional array of elements 72 disposed on the substrate structure 70 to form an FSS capable of reflecting at least a portion of energy of the first frequency incident on the case 68, while passing at least a portion of the energy of the second frequency to antenna. The FSS may be similar to the Type A FSS illustrated in FIG. 5a or the Type B FSS illustrated in FIG. 5b.

As another example, referring to FIG. 10, each of the stimulation leads 12 may comprise an outer layer 78 (or housing), which includes a tubular substrate structure 80 and a two-dimensional array of elements 82 disposed on the substrate structure 80 to form an FSS capable of reflecting at least a portion of energy of the first frequency incident on the outer layer 78. The FSS may be similar to the Type A FSS illustrated in FIG. 5a.

Although the afore-mentioned technique has been described in the context of an MRI, it should be appreciated that this technique can be used to reflect other electromagnetic energy generated by any source that could be harmful to the patient or electronic componentry of the SCS system 10.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. An implantable medical device, comprising:
an antenna configured for wirelessly receiving energy of a first frequency from an external device;
electronic circuitry configured for performing a function in response to the receipt of the received energy; and
a biocompatible housing containing the electronic circuitry and antenna, the housing including a substrate structure and a two-dimensional array of elements disposed on the substrate structure, wherein the array of elements and substrate structure are arranged in a manner that creates a frequency selective surface capable of reflecting at least a portion of energy of a second frequency incident on the housing, while passing at least a portion of energy of the first frequency incident on the housing to the antenna.

2. The implantable medical device of claim 1, wherein the function is programming the implantable medical device.

3. The implantable medical device of claim 1, wherein the function is charging the implantable medical device.

4. The implantable medical device of claim 1, wherein the transmission coefficient for the energy of the first frequency incident on the housing is greater than 0.5, and the reflection coefficient for the energy of the second frequency incident on the housing is greater than 0.5.

5. The implantable medical device of claim 1, wherein the transmission coefficient for the energy of the first frequency incident on the housing is greater than 0.75, and the reflection coefficient for the energy of the second frequency incident on the housing is greater than 0.75.

6. The implantable medical device of claim 1, wherein the second frequency is greater than 10 MHz.

7. The implantable medical device of claim 1, wherein the first frequency is less than 200 KHz.

8. The implantable medical device of claim 1, wherein one of the substrate structure and the array of elements is composed of a dielectric material, and the other of the substrate structure and the array of elements is composed of an electrically conductive material.

9. The implantable medical device of claim 8, wherein the one of the substrate structure and the array of elements is the substrate structure, and the other of the substrate structure and the array of elements is the array of elements.

10. The implantable medical device of claim 8, wherein the one of the substrate structure and the array of elements is the array of elements, and the other of the substrate structure and the array of elements is the substrate structure.

11. The implantable medical device of claim 8, wherein the electrically conductive material is metal, and the dielectric material is ceramic or plastic.

12. The implantable medical device of claim 1, wherein the array of elements is periodic.

13. The implantable medical device of claim 1, wherein the elements have the same shape.

14. The implantable medical device of claim 1, wherein each of the elements is one of a linear dipole, a crossed dipole, a loop, and a bow-tie.

15. The implantable medical device of claim 1, wherein each of the elements comprises an impedance load.

16. The implantable medical device of claim 15, wherein the impedance load is adjustable between a first value and a second value, the implantable medical device further comprising an electronic controller coupled to the impedance load, the electronic controller configured for generating a signal that dynamically adjusts the impedance load between the first value and the second value, such that the frequency selective surface reflects a portion of the energy at the second frequency incident on the housing when the impedance load has a first value and reflects a portion of the energy at a third frequency incident on the housing when the impedance load has a second value.

17. The implantable medical device of claim 1, further comprising a battery contained within the housing, the battery including another substrate structure and another two-dimensional array of elements disposed on the other substrate structure, wherein the other array of elements and other substrate structure are arranged in a manner that creates a frequency selective surface capable of reflecting at least a portion of energy of a third frequency incident on the battery, while passing at least a portion of the energy of the first frequency incident on the battery to the antenna.

18. The implantable medical device of claim 1, further comprising a lead coupled to the electronic circuitry, the lead including a tubular substrate structure and another two-dimensional array of elements disposed on the tubular substrate, wherein the other array of elements and other substrate structure are arranged in a manner that creates a frequency selective surface capable of reflecting at least a portion of energy of a third frequency incident on the lead.
